(19) United States
(12) Patent Application Publication
(10) Pub. No.: US 2025/0009264 A1
(43) Pub. Date: Jan. 9, 2025

(54) MICRONEEDLE PATCH DEVICE WITH INTEGRATED BIOELECTRODE

(71) Applicants: COMMISSARIAT A L’ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITE GRENOBLE ALPES, Saint-Martin-d’Heres (FR)

(72) Inventors: Bastien DARMAU, Saint-Martin-d’Heres (FR); Andrew GROSS, Gieres (FR); Isabelle TEXIER-NOGUES, Grenoble Cedex 09 (FR)

(73) Assignees: COMMISSARIAT A L’ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITE GRENOBLE ALPES, Saint-Martin-d’Heres (FR)

(21) Appl. No.: 18/764,529

(22) Filed: Jul. 5, 2024

(30) Foreign Application Priority Data
Jul. 6, 2023 (FR) 2307216

(51) Int. Cl. A61B 5/1473; A61B 5/00; A61B 5/145; A61L 31/04; A61L 31/14

(52) U.S. Cl. CPC A61B 5/14735; A61B 5/14514; A61B 5/14546; A61B 5/6833; A61B 5/685; A61L 31/041; A61L 31/145; A61B 2562/12

(57) ABSTRACT

A device for transdermal electrochemical measurements, comprising a polymeric support having a surface provided for contact with skin, an array of polymeric microneedles integral with the support and projecting outward from the surface of the support, and a porous and/or nano/micro-structured bioelectrode. A biologically active species is immobilized on the surface of a conductive material. The microneedles and a surface of contact of the support with the skin are formed of a biocompatible crosslinked hydrogel, non-electron-conductive in the dry state and electrolyte-conductive upon contact with an aqueous fluid. The bioelectrode is arranged in contact with the hydrogel and is free of direct contact with the skin. Methods for preparing and using the device are also described.

### TECHNICAL FIELD

The present invention relates to the field of devices of use for transdermal characterization of biomarkers and analytes in interstitial fluids. In particular, such a device is in the form of a microneedle patch coupled to an electrode comprising at least one biologically active species, preferably an enzyme, and called a bioelectrode. In order to limit repetitive, invasive and painful blood sampling and to afford more comfort to patients, the development of in/on-vivo transdermal devices has attracted considerable attention in recent decades for wearable and rapid personal health monitoring. These devices are based on the detection or quantification of biomarkers and analytes in interstitial fluids (ISF) as a suitable source of biological information for better understanding and management of human health. ISF is a natural body fluid present between tissues and cell and comprises electrolytes, nutrients and many other biomarkers. ISF is also abundant in the human body (about 15-25% of human body weight), is located just below the skin in regions with fewer nerves, such as the dermis or hypodermis, and is therefore easily accessible. The metabolite composition of ISF is often closely correlated with that of blood. For example, for glucose biodetection, the glucose level in the ISF is about 80 to 90% of the blood glucose concentration (about 5 mmol.1⁻¹). Other biomarkers and analytes present in the ISF at concentrations nmol.1⁻¹ to mmol.1⁻¹ and relevant for electrochemical biosensors may include, for example, lactate, alcohol, nitrate, alanine, cysteine, pyruvate, glycerol, Ca²⁺, Mg²⁺, K⁺, phosphate, urea, cholesterol, glutamate.

Wearable devices for continuous glucose monitoring, or CGM devices, are commercially available and have already revolutionized the monitoring and management of glucose levels in ISF. These electrochemical devices are based on the use of a needle for collecting ISF from the subcutaneous tissues. Enzymatic amperometric glucose biosensors are the most common type of commercially available biosensors, thanks to advantages that include the high reactivity and the high specificity of the enzymes for enzymatic and electroenzymatic reactions. Despite better management of diabetes, significant disadvantages of these wearable devices include the needle length, which is typically between 5 and 7 mm and can cause inconvenience to patients, and the sensor itself, which has limited lifetime, accuracy and precision. There are also other technical constraints, for example linked to the size and adhesive footprint of the device, to the need to use expensive redox membranes or mediators (biocatalytic components for the biosensor), or to limited biocompatibility and biodegradability.

Microneedle patches, or MN patches, have become one of the most promising alternatives for replacing the uncomfortable or invasive needles used in CGM devices. MN patches are arrays of needles of micrometric size with heights of generally between 20 and 2000 μm which are capable of penetrating specifically the cutaneous barrier in order to reach the dermis layer and access the dermal ISF. Located above the hypodermis, the dermis has fewer nerves and vessels, which makes transdermal MN patches minimally invasive and less painful, due to their small dimensions, free of blood and and less prone to causing infections. Nevertheless, the ability to offer such advantages requires careful consideration in the design of MN patches, related to factors such as the materials used, their geometry. and the way in which the microneedles are applied.

Like the commercial CGM devices of the needle type, the biosensors integrated in or on an MN patch are mainly based on electrochemical enzymatic biosensors, in particular because of their selectivity, specificity, low cost and simplicity of manufacture. MN patches are generally prepared by micro-machining, lithography, etching, printing and micro-molding processes and are manufactured using polymers and/or metals and/or ceramics. The different types of MNs are solid, hollow and porous MNs.

Solid MNs with integrated biosensor are constructed on the basis of a hard support, e.g. polycarbonate, [Ref. 1] Damien K Ming et al BMJ Innov, 2022, 8, 87-94, poly(methyl methacrylate) (PMMA), and the bioelectrode on the basis of a thin conductive metal (for example Au, Pt) on which are deposited biocatalytic components (enzyme, redox mediator, etc., sometimes included in a porous polymer matrix) and sometimes coated with biocompatible polymer layers or membranes for reasons of biocompatibility and porosity. The bioelectrode is formed directly on the external surface of the MNs, which thus comprises the MN support (made of metal or rigid polymer such as polycarbonate) and then a layer with the biocatalytic components (redox mediator and enzyme), and optionally a membrane or a layer made of biocompatible polymer. Such solid MNs have been used for the detection and/or monitoring of glucose and other metabolites. Unfortunately, direct contact between the sensor electrode and the tissues and biological fluid promotes cytotoxicity and stability problems. In addition, a delamination of the materials constituting the bioelectrode may occur upon insertion of the MN device into the skin (i.e. mechanical damage to the electrode layer, generating residual metal debris left in vivo).

For the MNs referred to as hollow, the enzymatic bioelectrode sensor is integrated either (i) inside the channel of a hollow needle of the MN patch [Ref. 2] Hazhir Teymourian et al. Anal. Chem., 2020, 92, 2, 2291-2300, or (ii) on the back of the device [Ref. 3] US 20090099427A1. Despite generally longer detection times than for the solid MN biosensor, due to the driving force (e.g. capillary forces) required to establish contact between the biosensor and the ISF/biomarkers, this approach may offer efficient fluid extraction and flexibility in terms of the type of bioelectrodes that can be incorporated. However, leaching of the biocatalytic components into the ISF is still a problem. There are also limitations in terms of achieving effective penetration because the needles are less mechanically resistant than solid MNs and do not have an ideal geometry for skin penetration.

MNs made of porous materials (for example based on poly(glycidyl methacrylate) (PGMA) or silk) are a hybrid type between solid and hollow MNs. [Ref. 4] Hiroyuki Kai et al., J. Phys. Energy, 2021, 3, 024006. The MNs can be considered sufficiently strong to ensure effective penetration into the skin, while providing a pore structure, if necessary conductive because it is metallic [Ref. 4], suitable for (i) depositing on the pores enzymes and mediators constituting a bioelectrode and necessary for electrochemical detection, and (ii) improving the contact of the ISF with enzymes and mediators and the conductive surface constituting the bioelectrode of a sensor. The ISF can be guided to the biosensor via capillary forces, as is typically the case for hollow MNs.

Unfortunately, all of these integrated MN-enzymatic electrode biosensors (bioelectrodes) have the disadvantage of requiring costly and time-consuming manufacturing processes and of being compatible only with complex and often limited electrode integration processes. However, for obvious reasons, these costly and complex integration methods are an obstacle to their industrial production. In addition, all the MN devices for which the electrode materials are deposited on the external surface of the MN are subject to considerable toxicity problems linked to the sensor, for example in the event of the coating breaking or coming loose during piercing of the skin and/or operation of the appliance, or simply because of direct contact between the coating and the skin tissues and fluids. Although hollow or porous MNs with the electrode behind the microneedles are more flexible, in terms of the type of electrodes that can be integrated, and can offer improved safety, they require an increased latency time and forced ISF extraction. Moreover, a common problem lies in obtaining good adhesion of the electrode to the MN device. [Ref. 7] SeungHyun Park et al. Biosensors and Bioelectronics, 220, 2023, 1149121

Consequently, there remains a need for a device which is of use for transdermal electrochemical measurement, coupling at least microneedles with at least one bioelectrode, and of which the manufacture is not complex.

There is also a need to ensure that the use of this device does not pose a risk of toxicity with regard to the subject on whom the analysis is performed.

There is also a need for such a device to be compatible with the use of a wide variety of bioelectrodes.

There is also a need to ensure that such a device allows good adhesion of this bioelectrode and, in particular, that the bioelectrode is not subject to a detachment phenomenon.

There is also a need for this bioelectrode to have rapid contact with the ISF to be characterized, without in so doing having contact with the skin.

The present invention aims precisely to meet these needs.

### SUMMARY OF THE INVENTION

Thus, according to one of its aspects, the present invention relates to a device of use for transdermal electrochemical measurements, said device comprising at least one polymeric support 1 having a surface provided for contact with skin 2, at least one array of polymeric microneedles 3 integral with said support 1 and projecting outward from said surface of the support 1 provided for contact with said skin 2, and at least one porous bioelectrode 10, optionally nano- or micro-structured, comprising at least one biologically active species, in particular an enzyme, immobilized on the surface of a conductive material, characterized in that said microneedles 3 and at least the surface of contact of said support 1 with said skin 2 are formed of a biocompatible crosslinked hydrogel, non-electron-conductive in the dry state and electrolyte-conductive upon contact with an aqueous fluid, and in that said bioelectrode 10 is arranged in contact with the hydrogel provided to swell upon contact with said aqueous fluid and is free of direct contact with the skin 2.

Within the meaning of the invention, a porous bioelectrode is a bioelectrode capable of being interpenetrated by the hydrogel at least at the surface and, where appropriate, at depth. Thus, according to a particular embodiment, a bioelectrode can be formed of a conductive material which is porous, in particular chosen from the group consisting of platinum, platinum-iridium, gold, palladium, iridium, their alloys, graphite, carbon, indium tin oxide, ruthenium dioxide, conductive polymer or carbon nanotubes.

According to another embodiment, a bioelectrode can comprise, on the surface of its conductive material and in contact with the one or more immobilized biological species, one or more porous polymeric materials chosen, for example, from poly(ethylene oxide), polymers with heterocyclic nitrogen groups, for example poly(vinylpyridine) or poly(vinylimidazole), perfluorinated ionomer polymers, for example Nafion, poly(urethane), redox polymers (i.e. polymers with multiple redox centers based on transition metals such as osmium, a polysaccharide biopolymer, for example cellulose acetate, dextran or chitosan, a conductive polymer based on poly(aniline) or poly(3,4-ethylenedioxythiophene) derivatives, or a combination thereof. Thus, in this second embodiment, the conductive material of the bioelectrode 10 may or may not be natively porous.

Unexpectedly, the inventors have thus found that the coupling of a porous and optionally nano-structured or micro-structured bioelectrode to a support comprising at least one array of microneedles can be performed under conditions allowing the abovementioned expectations to be met, provided that this support is formed in particular of a hydrogel according to the invention, namely capable of changing from a non-electron-conductive state when in a dry state to an electrolyte-conductive state when swollen by an aqueous fluid. A porous aspect of the bioelectrode moreover has the advantage of allowing penetration of said hydrogel into the pores of the bioelectrode and of thus promoting intimate contact between the surface of the bioelectrode on which is immobilized at least one biologically active species, preferably an enzyme, if necessary in combination with an electrochemical mediator, and the interstitial fluid that has soaked the hydrogel. This intimate contact promotes the physical attachment between the bioelectrode and the constituent polymer of the substrate or the MN patch needles, and it also promotes the performance of the biologically active species and the bioelectrode.

More precisely, and as will be seen from the examples below, the microneedles 3 of the device according to the invention advantageously possess, in the dry state, the mechanical strength necessary for perforating a body tissue, in particular to depths of between 200 and 2000 μm in the case of human skin, in order to access its interstitial fluid (ISF) efficiently. When the crosslinked and dry hydrogel constituting these microneedles 3 is in contact with this ISF, it swells, and the fluid to be analyzed will diffuse there by difference in osmotic pressure and/or capillarity. Thus, from an electrical point of view, the microneedles 3 and the part of the support 1 integral with its microneedles 3 can be considered, when they are impregnated with ISF, as becoming an extension of the tissues with the ISF and then perform an electrolyte function. The bioelectrode comprising the biologically active species placed in contact with this hydrogel swollen with ISF makes it possible, under these conditions, to carry out a measurement or operation faithful to that which would be carried out if it were inserted directly into the skin, but advantageously in a minimally invasive manner. Moreover, the integration method described in the invention makes it possible to take advantage of the porosity of the bioelectrode 10. By interpenetrating at the level of the bioelectrode 10, the hydrogel maximizes the contact surface between the one or more active species of the bioelectrode 10 and the interstitial fluid conveyed by the hydrogel during its swelling.

According to a variant embodiment, the or at least one bioelectrode 10 is integrated in the crosslinked hydrogel constituting the support 1 which is in direct contact with the rear of the microneedles 3.

According to another variant embodiment, the or at least one bioelectrode 10 is integrated in the crosslinked hydrogel constituting the microneedle(s) 3.

In particular, the bioelectrode 10 is at least partially interpenetrated by crosslinked hydrogel.

According to yet another variant embodiment, the device according to the invention comprises at least one auxiliary electrode, distinct or not from a bioelectrode. In particular, the device according to the invention can comprise at least one auxiliary electrode distinct from a bioelectrode, in particular chosen from a reference electrode, a counter-electrode, a hybrid reference electrode and counter-electrode.

In such an embodiment, the device according to the invention can comprise at least one of the electrodes, in particular a porous bioelectrode 10, in the constituent crosslinked hydrogel of the support 1 in direct contact with the rear of the microneedles 3, and at least one or more other electrodes in the constituent crosslinked hydrogel of the microneedles 3.

The device according to the invention is therefore advantageous in several respects.

Its constituent crosslinked hydrogel is compatible with the integration of any bioelectrode 10 for the detection of analytes, ions and/or biomarkers. This hydrogel provides in addition to an enzymatic bioelectrode a suitable aqueous environment to operate under satisfactory conditions and in particular prolongs the life/stability of the bioelectrode.

Only its crosslinked hydrogel component is in direct contact with the biological tissue being analyzed, and therefore the risk of delamination of the coating and/or constituent material of the bioelectrode (e.g. mediator, enzyme, AgCl layer, etc.) upon insertion of the microneedles into the tissue is avoided.

The risk of detachment of a component or components of the bioelectrode 10 is also avoided on account of the integration therein at depth of the crosslinked hydrogel of the device according to the invention.

The positioning of the bioelectrode 10 close to the rear of the tips of the microneedles 3 and/or in microneedles 3 themselves, also limits the distance that the analyte, ion, proton and/or biomarker must travel and/or any mass transport limitation that could affect detection performance, including reduced latency time.

According to a particular variant, the device comprises at least one bioelectrode 10 comprising an enzyme, in particular an oxidoreductase.

In particular, the bioelectrode 10 is of the electrochemical glucose biosensor type, especially of the 2nd generation type, which combines the enzyme flavin adenine dinucleotide-dependent glucose dehydrogenase (FAD GDH) with a redox mediator, 1,10-phenantholine-5,6-dione (PLQ), physically adsorbed at the level of a porous electrode made of multi-walled carbon nanotubes (MWCNT), in particular as described in the document WO2018 115710A1.

According to a particular variant, the device according to the invention is in the form of a transdermal microneedle (MN) patch. In particular, the polymeric microneedles (3) are not hollow.

According to a preferred variant, the assembly composed of support 1 and microneedles 3 is formed of said biocompatible crosslinked hydrogel which is not electron-conductive in the dry state.

In particular, the crosslinked hydrogel is free of any metallic constituent and electron-conductive polymer.

According to another preferred variant, the hydrogel is free of biologically active species other than that/those immobilized on said bioelectrode(s) 10.

According to another preferred variant, said biologically active species is an enzyme, in particular an oxidoreductase, and participates in an electrocatalytic reaction with an exchange of electrons between the enzyme and the conductive material.

According to another of its aspects, the present invention relates to a method for preparing a device according to the invention, in particular by micro-molding, and comprising at least the crosslinking of at least one polymer, preferably a biopolymer, in order to form said crosslinked hydrogel, characterized in that at least said or one porous bioelectrode 10, optionally nano- or micro-nanostructured, is integrated by being placed in contact with said polymer prior to or simultaneously with its crosslinking.

In particular, said bioelectrode 10 and even the auxiliary electrodes are placed in contact with the aqueous formulation containing at least said non-crosslinked polymer and are subjected there to mechanical pressure in order to position them at depth in proximity to the base of the microneedles 3 and/or at depth in the microneedles 3, prior to or simultaneously with the crosslinking.

According to yet another of its aspects, the present invention relates to the use of a device according to the invention for transdermal electrochemical measurements, in particular for characterizing at least one analyte in an interstitial fluid.

Thus, the invention relates more particularly to a method for detecting and/or assaying at least one analyte in an interstitial fluid of a patient, in particular in a non-invasive manner, comprising at least placing the microneedles 3 of a device according to the invention in contact with said interstitial fluid of said patient under conditions that are conducive to the swelling of the constituent crosslinked hydrogel of the microneedles 3 by this fluid and to the diffusion of this fluid, by a difference in osmotic pressure and/or capillarity, as far as the bioelectrode(s) 10 of said device.

Other features, variants and advantages of the subjects of the invention will emerge more clearly on reading the description, the examples and figures which follow, which are given by way of illustration and do not limit the invention.

In the remainder of the text, the expressions “of between . . . and . . . ”, “ranging from . . . to . . . ” and “varying from . . . to . . . ” are equivalent and are intended to mean that the limits are included, unless otherwise stated.

### BRIEF DESCRIPTION OF THE DRAWINGS

FIG. 1 shows a schematic example of a device according to the invention.

FIG. 2 shows cyclic voltammograms recorded at a scanning rate=1 mV·s−1 in phosphate buffer (PB) (0.1 M, pH=7.4) with a crosslinked dextran-methacrylate (Dex-MA) microneedle biosensor with a degree of substitution (DS=9%), comprising an electrode based on MWCNTs coated with immobilized PLQ and FADGDH (MN-BS) in the absence (dotted line) of glucose and in the presence (solid line) of glucose 0.1 M.

FIG. 3 illustrates continuous glucose monitoring by chronoamperometry at −0.1 V versus AG/AgCl pseudo-reference with a microneedle biosensor of crosslinked Dex-MA (DS=37%) comprising an electrode based on MWCNTs coated with PLQ and FADGDH and immobilized (MN-BS) in an artificial skin containing 5 mmol.1−1 glucose, n=1.

FIG. 4 illustrates a selectivity test by chronoamperometry in PB at −0.1 V versus AG/AgCl pseudo-reference and with stirring at 300 rpm with a MWCNT biosensor without hydrogel coating and with hydrogel coating in the presence of 5 mmol.1-1 glucose (1), then successive additions of 2) 0.2 mmol.1-1 of acetaminophen, 3) 1 mmol.1-1 of cholesterol, 4) 8 mmol.1-1 of urea, 5) 2 mmol.1-1 of lactate, 6) 0.3 mmol.1-1 of galactose, 7) 0.5 mmol.1-1 of uric acid UA, 8) 0.1 mmol.1-1 ascorbic acid.

### DETAILED DESCRIPTION

As will be clear from the above, the invention aims to propose minimally invasive devices that can intervene transdermally, either continuously or non-continuously, for transdermal detection of biomarkers in interstitial fluids.

More particularly, this device comprises at least one polymeric support 1, at least one porous bioelectrode 10, and microneedles 3 arranged on one of the faces of the support 1. These microneedles 3 penetrate into a biological tissue, as far as the interstitial fluid thereof, in which it is precisely sought to electrochemically characterize at least one chemical or biological species.

In the device according to the invention, the microneedles 3 are solid. In other words, they are not hollow but are formed of crosslinked hydrogel in accordance with the invention and, where appropriate, of a bioelectrode 10 integrated into this hydrogel but without any possible direct contact with the tissue that is being analyzed.

They generally have a length varying from 200 to 3000 μm.

In a variant embodiment, the device according to the invention can comprise several arrays of microneedles 3 and/or several bioelectrodes 10 and/or distinct electrodes.

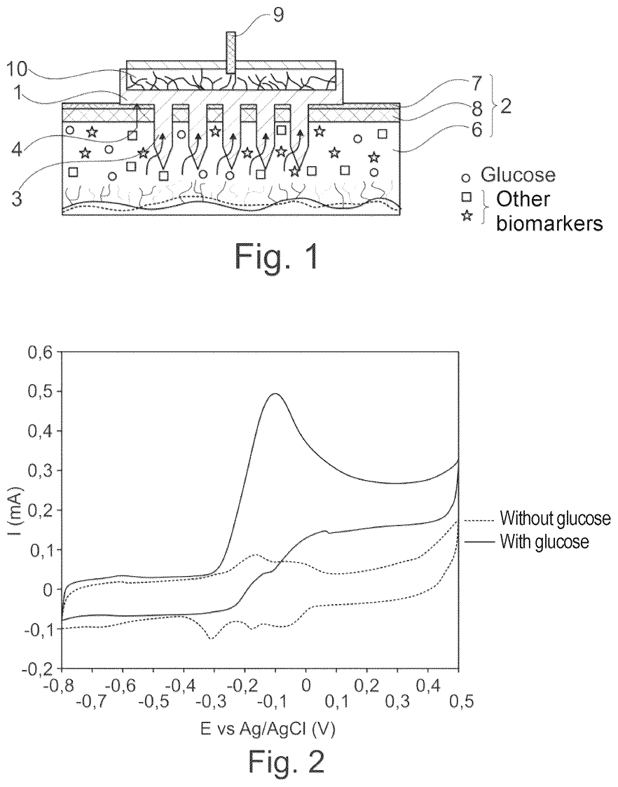

In particular, the device according to the invention is in the form of a microneedle (MN) patch, in particular as illustrated in FIG. 1.

The device in FIG. 1 is formed by a body made of a crosslinked hydrogel which, when not in use, is in a dry state. The body comprises a support 1, and microneedles 3 which project from a face 4 of the support 1 in contact with the skin 2 of a patient and which have penetrated into the dermis 6 of the skin of the patient, through the stratum corneum 7 and the epidermis 8.

The device further comprises a bioelectrode 10. This bioelectrode 10 is porous, which means that the hydrogel can interpenetrate the structure of the electrode (as is shown in FIG. 1). The interstitial fluid present in the dermis comprising the biomarkers diffuses, thanks to the hydrogel, toward the bioelectrode 10 through the microneedles 3 then into the support 1, and finally into the pores of the porous bioelectrode 10, forming a crosslinked hydrogel filled with electrolyte (interstitial fluid).

An electrical connection 9 of the bioelectrode can be effected by a wire made of an electrically conductive material (for example a silver plated copper wire) adhering to the back of the bioelectrode with a conductive paste (such as carbon paste).

### Crosslinked Hydrogel

Within the meaning of the invention, a crosslinked hydrogel is a hydrophilic and insoluble three-dimensional polymer. Its crosslinking is obtained by physical or chemical bonds, preferably chemical bonds, existing between polymer chains.

In the dry state, the crosslinked hydrogel possesses the hardness necessary for the penetration of the microneedles 3 of which it is composed, but on the other hand it is not capable of ensuring circulation of electrons. In other words, it is not an electron conductor. It is thus without a metallic constituent and organic conductive polymer such as PEDOT, poly(pyrrole) or poly(aniline).

Moreover, this dry crosslinked hydrogel is capable of swelling upon contact with a fluid, especially an aqueous fluid such as water or a biological fluid. It can therefore absorb and retain a large amount of a fluid. This hydrogel swelling rate (SR) is generally defined by the ratio between the weight of the swollen hydrogel and the weight of this same hydrogel in the corresponding dry state. More precisely, the fluid diffuses inside the crosslinked hydrogel by capillary phenomena and/or osmotic pressure difference between the fluid and the hydrogel. Thus impregnated, the crosslinked hydrogel makes it possible to bring the aqueous fluid to be characterized into contact with, in particular, the bioelectrode that it integrates. In particular, the hydrogel, by at least partially penetrating the porous bioelectrode, increases the contact surface between the fluid and the bioelectrode. In other words, the swelling of the hydrogel by the interstitial fluid causes the device according to the invention, insulating in the dry state, to become an electrolytic matrix (ionic conductor) and makes it possible to form the electrical bridge between the tissue where the microneedles 3 are inserted and the one or more bioelectrodes, and possibly electrodes, which it contains. In contrast to devices of the prior art, the crosslinked and swollen hydrogel according to the invention also acts as a filter isolating the one or more bioelectrodes/electrodes, which it integrates, from any possible contact with the biological tissue. The structural network of the crosslinked gel is such that, in the swollen state, it makes it almost impossible for any small fragments of the one ore more bioelectrodes/electrodes to migrate toward this tissue. Any risk of toxicity due to the bioelectrodes/electrodes is therefore excluded.

The crosslinked hydrogel according to the invention is also biocompatible.

Within the meaning of the invention, a biocompatible hydrogel is a hydrogel which, by its chemical nature, is not such as to cause an adverse effect in the biological tissue that is analyzed.

The biocompatible aspect of the hydrogel is an asset, because only it is in direct contact with tissues as a component of the microneedles 3 penetrating the skin of a subject. Thus, the hydrogel constituting the microneedles 3 and the support 1 acts as a screen between the tissue and the bioelectrode 10 and any auxiliary electrodes associated with it in the device according to the invention. Moreover, if the hydrogel, although crosslinked, remains bioresorbable over the long term, the hydrogel components of the polymer microneedles 3 will not pose any risk if a microneedle breaks in the skin 2 during use of the device, since they will be able to be resorbed over time without damaging the tissues.

Finally, in view of its biocompatibility, the hydrogel can play a beneficial role as regards the stability of associated bioelectrode(s) 10 in the device according to the invention. It constitutes a biocompatible environment, in particular one very advantageous for the enzymes, makes it possible to reduce their denaturation and can allow favorable mass transport.

As has already been specified, the crosslinked hydrogel according to the invention advantageously possesses good swelling properties (swelling in 24 hours greater than 10%, preferably greater than 20%, more preferably more than 50%) and, in the dry state, good mechanical hardness for piercing the skin (Young's modulus greater than 10 kPa).

As is set out in detail below, the precursor polymer of this hydrogel must also be in the “liquid” state for the integration of at least one bioelectrode 10 or even auxiliary electrodes and make it possible to penetrate at least partially into the structure of said bioelectrode. The polymer solution can also be solid at ambient temperature but liquid at a temperature compatible with the one or more biologically active species, in particular the enzyme or enzymes, associated with the bioelectrode (for example <60 degrees or optionally <80 degrees for thermostable engineered enzymes). This is the case, for example, with gelatin gels.

The crosslinked hydrogel according to the invention can be obtained by crosslinking any type of polymer which, once crosslinked, forms, in contact with an aqueous fluid, an insoluble and infusible hydrogel.

In particular, the crosslinked hydrogel according to the invention can be obtained by crosslinking one or more biopolymers with, where appropriate, one or more synthetic polymers, where appropriate chemically modified to be crosslinkable, or a mixture thereof. Within the meaning of the invention, the term “biopolymer” denotes a polymer derived from renewable natural resources (a material also referred to as “natural”). The biopolymers considered according to the invention have the advantage of possessing, in addition to very good compatibility with human skin, biodegradability as such or even in vivo.

This term “biopolymer” thus covers natural polymers such as, for example, a protein or a polysaccharide, but also derivatives of natural polymers such as, for example, those derived from a chemical modification of these natural polymers to make them crosslinkable or compatible with a specific crosslinking mode.

This chemical modification usually consists of introducing, into the structure of the polymers to be modified, crosslinkable units, in particular chosen from thiol, divinylsulfonyl, maleimide, azide, alkynyl, alkenyl, acrylate, methacrylate, aldehyde, norbornenyl, oxy-amine, and in particular from acrylates, methacrylates.

By way of illustration of synthetic polymers suitable for forming a crosslinked hydrogel suitable for the invention, mention may be made in particular of crude poly(ethylene glycols) (PEG) or those modified by different chemical functions, for example poly(ethylene glycol) diacrylic (PEGDA), poly(ethylene glycol) dimethacrylic (PEGDMA), poly(vinyl pyrrolidone) (PVP), poly(methyl vinyl ether maleic acid)-PEG, PVA-MA,-PLA,-PLLA,-PGA,-PLGA and their derivatives.

By way of illustration of biopolymers suitable for forming a crosslinked hydrogel in accordance with the invention, mention may be made in particular of polyhydroxy acids, for example polyglycolic acid (PGA) and polylactic acid (PLA), also known as polylactide; polysaccharides, for example cellulose, chitin and starch, and proteins or peptides, for example collagen, gelatin, elastins, fibroins, maize zein, the silk of various species of silkworms, keratin and their derivatives.

According to a preferred variant embodiment, the crosslinked and biocompatible hydrogel considered according to the invention is obtained by crosslinking at least one biopolymer chosen from polymers such as chitosan, dextran, alginates, collagen, gelatin, agarose, chitin, polyhydroxyalkanoates, pullan, starch, amylose, amylopectin, cellulosic, in particular carboxymethylcellulose, and hydroxypropylcellulose, hyaluronic acid, gellan gum, xanthan gum, for example the derivatives of these polymers resulting from their chemical modification to make them crosslinkable or compatible for a specific crosslinking mode, and combinations thereof.

According to a particular embodiment, this biopolymer is or derives from at least one polymer chosen from alginates, hyaluronic acid, carboxymethylcellulose, chitosan, dextran and derivatives thereof.

In particular, the hydrogel is a crosslinked dextran hydrogel.

Within the meaning of the invention, the term dextran covers dextrans which have or have not been chemically modified in order to be crosslinked.

Thus, it may be advantageous to crosslink a dextran polymer chemically modified by acrylate and/or methacrylate units which are well known to be photopolymerizable.

These functional groups, which make it possible to crosslink by irradiation, are introduced with a given degree of substitution, DS. Conventionally, the DS corresponds to the number of polymerizable functional groups introduced on 100 monomer units. This DS can be determined by 1H NMR.

Thus, according to a particular embodiment, the device according to the invention comprises a hydrogel derived from the crosslinking of a dextran polymer, modified by units chosen from acrylate, methacrylate, alkenyl and alkynyl, preferably a dextran polymer modified by methacrylate units, Dex-MA, and of DS varying from 5 to 65%.

Electrodes and their Mode of Integration

Another specific feature of the device of the invention lies in the mode of integration of the one or more porous bioelectrodes required according to the invention or of other auxiliary electrodes.

As has been specified above, the electrodes are arranged so that they do not have direct contact with the tissue of which the ISF is being analyzed.

In general, they are fully integrated into the crosslinked hydrogel to be swollen by this ISF, with the exception of their end provided for the external electrical connection. Generally, this connection is located on the upper face or one of the lateral faces of the device.

Within the scope of the present invention, and in contrast to conventional devices, the bioelectrode(s) and possibly the auxiliary electrode(s) are also not in communication with a channel to which the microneedles would be connected.

As has already been specified above, they are instead disposed either close to and/or within solid microneedles of the device.

According to a particular embodiment, they are integrated within the thickness of the support located to the rear of the microneedles.

This mode of integration is illustrated in particular in FIG. 1.

### Bioelectrodes

Bioelectrodes are electrodes widely described in the literature, and persons skilled in the art are able, based on their general knowledge, to access bioelectrodes that satisfy the specificities of the invention. The term “bioelectrode” very often refers to an electrode comprising one or more enzymes.

In particular, a bioelectrode according to the invention can comprise a conductive material chosen from electrodes based on Pt, Au, Ag, Pg, Ni, Ir, graphitic carbon, amorphous carbon, graphene, graphene oxide, diamond, boron-doped diamond, nanotubes, doped semiconductor fibers, e.g. doped silicon, metal oxides, e.g. indium tin oxide, conductive polymer electrodes, for example a PEDOT material, and conductive fibers.

A bioelectrode can be nano-structured or micro-structured. It can thus be a nano- or micro-electrode.

Its general shape may or may not be planar. A planar bioelectrode can be an electrode printed on a porous or non-porous substrate. This substrate can comprise a sheet of carbon paper or of tissue or a sheet or pad or a printed electrode, e.g. a screen-printed electrode.

A bioelectrode of non-planar shape comprises a Pt wire or a carbon fiber wire or a wound electrode.

For example, the bioelectrodes suitable for the invention can be chosen from bioelectrodes based on Pt, Au, Ag, Pd, Ni, Ir, graphitic carbon, amorphous carbon, graphene, graphene oxide, diamond, boron-doped diamond, nanotubes, doped semiconductor fibers, e.g. doped silicon, metal oxides, e.g. indium tin oxide, conductive polymer electrodes, e.g. PEDOT, and conductive fibers.

When the conductive material of the bioelectrode according to the invention is non-porous, it is surface-modified by a polymeric or inorganic porous material. This material may in particular be chosen from crosslinked hydrogels or polymers, for example Nafion, a cellulose acetate, a sulfonated poly(ether ketone), glutaraldehyde, a poly(ethylene glycol) diglycidyl ether, poly(dimethyl siloxane), chitosan, a dextran, an alginate, redox polymers based on transition metal compounds and complexes, poly(ethylene oxide), polymers with heterocyclic nitrogen groups such as poly(vinylpyridine) or poly(vinylimidazole), poly(urethanes), conductive polymers based on poly(aniline) or poly(3,4-ethylenedioxythiophene) derivatives, and combinations thereof or copolymers.

It should be noted that such a material is also advantageous for improving the performance of the bioelectrode in terms of stability or the ability to detect interfering molecules or species. It is therefore possible to consider it in association with a porous conductive material.

According to another variant, the bioelectrodes 10 considered according to the invention are formed of a nano-structured or micro-structured conductive material.

They can thus be porous 3D bioelectrodes such as “papers” made up of carbon nanotubes and micro/nano-structured 2D bioelectrodes

In particular, the bioelectrode is based on carbon nanotubes and in particular based on multi-walled carbon nanotubes (MWCNT).

At least one biologically active species, in particular an enzyme, is immobilized on the surface of the conductive material.

Advantageously, the bioelectrode comprises at least one enzyme, if necessary combined with a molecule that facilitates electron transfer between the enzyme and the electrode 10, for example an electrochemical mediator or a molecule of the “promoter” type that improves the orientation and/or the transfer of the electrons between the electrode and the enzyme. On the other hand, the hydrogel of said device is advantageously free of any biologically active species or mediator, other than that/those immobilized on said bioelectrode(s).

Thus, the device can integrate a bioelectrode on which there are immobilized, generally by adsorption, one or more enzymes chosen in particular from oxidases, for example glucose oxidase, pyruvate oxidase, xanthine oxidase, lactate oxidase, dehydrogenases, for example lactate dehydrogenase, reductases, for example nitrate and nitrite reductase, and metalloenzymes, for example bilirubin oxidase or laccase, etc.

A bioelectrode can thus represent a working electrode (WE). The term “working electrode” here designates an electrode to which a candidate compound (analyte, biomolecule, active agent) is electro-oxidized or electro-reduced with or without the intervention of a redox mediator or “promoter” molecule. The term “redox mediator” designates an electron transfer agent that transfers electrons between a compound and an electrode directly or indirectly. The term “promoter” designates an agent that facilitates the orientation of enzymes on the surface and/or the transfer of electrons between the active site of the enzyme and the electrode.

In a particular embodiment, the device according to the invention comprises an enzymatic bioelectrode.

The bioelectrode can also be modified with a biological (or non-biological) catalyst in order to improve its performance, e.g. selectivity, specificity, activity.

According to yet another variant, the device can integrate a bioelectrode on which there is immobilized a non-enzymatic catalyst, for example conductive nanoparticles, for example Au, Pt, Ir and/or other nanostructured materials, e.g. carbon nanotubes, quantum dots, mesoporous and doped carbon, and/or redox molecules such as organic molecules or organometallic species.

According to yet another variant, the bioelectrode according to the invention can also be modified by a biorecognition element, for example streptavadin, extravidin, aptamers, etc. Such bioelectrodes are particularly advantageous for detecting and/or assaying a chemical or biological analyte, for example proteins, amino acids, viruses, hormones, medicaments, drugs (e.g. cannabinoids, amphetamines, cocaine and opioids) and nicotine metabolites (e.g. cotinine), and in particular an analyte chosen from glucose, lactate, alcohol, nitrate, alanine, cysteine, pyruvate, glycerol, Ca²⁺, Mg²⁺, K⁺ ions, phosphate, urea, cholesterol, glutamate, hydrogen peroxide, hydrogen, etc. This type of bioelectrode is often metal or carbon based and is used for electro-oxidation or electro-reduction of the candidate compound and often with one or more catalytic or electrocatalytic elements such as noble metals, nanoparticles, and enzymes. They can in particular be working electrodes (WE) with recognition elements (often called “receptors”) to facilitate specific and selective detection, and often with a polymeric element to facilitate properties such as mechanical, recognition and/or signal transmission properties.

Thus, as has already been specified above, a device according to the invention advantageously comprises at least one enzymatic bioelectrode which is provided in particular to interact with glucose and which associates the FAD-GDH enzyme with the redox mediator phenanthroline quinone (PLQ) physically adsorbed at the level of an electrode formed of multi-walled carbon nanotubes (MWCNT), in particular as described in the document WO2018 115710A1.

In another example, a device according to the invention can comprise at least one bioelectrode with an immobilized enzyme and at least one immobilized redox species chosen from a compound of osmium, ruthenium, iron and cobalt, coupled to a polymer chosen from poly(vinylpyridine), poly(aniline), poly(thiophene), poly(acetylene), poly(pyrrole), a biopolymer based on polysaccharides, or from aromatic molecules chosen from the group consisting of 9,10-phenanthrenequinone, 1,10-phenanthroline-5,6-dione, 9,10-anthraquinone, phenanthrene, 1,10-phenanthroline, 5-methyl-1,10-phenanthroline, phenazines, phenathiozines, tetrathiofulvalene, or inorganic complexes of an osmium, ruthenium, iron and cobalt compound, or polyoxometallates. The immobilized redox species will serve to improve the electrocatalytic or bioelectrocatalytic reaction.

Of course, a device according to the invention can integrate a plurality of bioelectrodes which are each able to interact with distinct biological species also known as biomarkers, such as lactate and glucose. The integration of a plurality of bioelectrodes with different sensors within the same device has the advantage of making it possible, for example, to monitor a plurality of biomarkers in interstitial fluids simultaneously and in real time. In another variant embodiment, a second bioelectrode provided for the same target can be used for a duplicate measurement or as a calibration electrode.

In a variant, the device according to the invention comprises at least one auxiliary electrode, distinct from the bioelectrode, chosen in particular from a reference electrode, a counter-electrode, and a hybrid reference electrode and counter-electrode. The sensor then functions in such a way that a current is generated between the working electrode, represented by the bioelectrode, and the counter-electrode or hybrid electrode.

Thus, the device according to the invention can also comprise a counter-electrode (CE). Such electrodes are particularly advantageous for establishing a circuit with a working electrode to which the current is applied or on which the current is measured. Electrodes of Pt, Ag, AgCl or other metals (Ti, Au, Pd, tin), metal oxides (IrO₂), carbon electrodes (vitreous carbon), conductive polymer electrodes (PEDOT), are auxiliary electrodes typically used in electrochemistry.

The device according to the invention can also comprise at least one reference electrode (RE). Such electrodes are particularly advantageous for measuring or controlling the potential of an indicator or of the working electrode. Saturated calomel electrodes or silver chloride electrodes are reference electrodes typically used in electrochemistry.

The device according to the invention can also comprise an electrochemical detection device attached to said electrode(s) via electrical connections preferably surrounded by insulation. This detection device thus makes it possible to carry out measurements by potentiometry, cyclic voltammetry (CV), fast-scan cyclic voltammetry (FSCV), square wave voltammetry (SWV), pulsed voltammetry or chronoamperometry.

### Method for Preparing a Device According to the Invention

As has been specified above, the invention also relates to a method for preparing a device according to the invention, in particular by micro-molding, comprising at least one step of crosslinking at least one polymer, in particular a biopolymer, in order to form said crosslinked hydrogel, characterized in that said bioelectrode of said device is integrated by being placed in contact with said polymer prior to or simultaneously with its crosslinking. In particular, during the contacting of the bioelectrode or even the auxiliary electrodes, the polymer is in the form of a formulation which in particular is aqueous, liquid or semi-liquid, that is to say of viscous appearance, preferably having a viscosity varying from 1 to 100 Pa·s⁻¹ at 1 Hz with a shear stress of 0.2%.

This liquid formulation, preferably aqueous, contains said polymer and preferably at least one crosslinking agent.

The electrode or electrodes to be integrated are placed on the surface of this liquid formulation and are there subjected to mechanical pressure in order to position them at depth and close to the base of the microneedles or within the constituent hydrogel of the microneedles, prior to or simultaneously with the crosslinking. In fact, at this viscosity, the one or more electrodes with their contact can be easily inserted with a moderate force (slight pressure of the thumb, for example) in a precise and/or reproducible manner. They can be inserted such that the contact pick-up wires (or other electrical contacts) preferably exit the patch from the top and/or the side of the support integral with the microneedles.

Moreover, the liquid aspect of the polymer formulation at the time of insertion of the porous bioelectrode allows the polymer to penetrate into the electrode structure and to thus obtain a large surface of contact with the hydrogel that will carry the interstitial fluid.

After the insertion of the electrode(s), the crosslinking is carried out or continued until a dry crosslinked hydrogel is obtained. To do this, the crosslinking operation is generally followed by a drying operation.

The crosslinking can be a chemical crosslinking. In the latter case, a crosslinking agent is also present in the liquid formulation containing the polymer(s) and preferably the biopolymer(s) to be crosslinked. This often slow method of crosslinking is conducive to the integration of the electrode(s) during the crosslinking.

The crosslinking can also be triggered by a physical stimulus such as heat, gas exposure or irradiation (for example light, UV radiation, X-rays, gamma radiation, microwave irradiation).

According to a particular embodiment, the crosslinking is carried out photochemically.

Advantageously, the devices according to the invention can be prepared by a micro-molding technique using a silicone mold, itself obtained from a master model produced by micro-machining.

The liquid formulation, preferably aqueous, containing the biopolymer and preferably at least one crosslinking agent is poured into the mold obtained from this master model. After evaporation of the water or solvent from the solution, and crosslinking, this mold makes it possible to obtain the dry crosslinked hydrogel device, which can be removed from the mold.

### Use

A device according to the invention is advantageously of use for transdermal electrochemical measurements, in particular for characterizing at least one analyte in an interstitial fluid. Within the meaning of the invention, the term analyte covers any chemical or biological species such as a medicament, a bioactive agent, a metabolite or an endogenous biochemical product.

Any analyte present in the interstitial fluid of the skin can be characterized using the device according to the invention. Examples include, but are not limited to, glucose, sodium, potassium, alcohol, lactate (important for athletes), cortisol, urea, drugs (e.g. cannabinoids, amphetamines, cocaine and opioids) and nicotine metabolites (e.g. cotinine) and also the medicinal substances that a patient may be taking for one or more medical conditions.

Thus, the present invention also relates to a method for detecting and/or assaying at least one analyte in an interstitial fluid, in particular in a non-invasive manner, comprising at least placing the microneedles of a device according to the invention in contact with said interstitial fluid under conditions that are conducive to the swelling of said constituent hydrogel of the microneedles by said fluid and to the diffusion of this fluid, by a difference in osmotic pressure and/or capillarity, as far as the bioelectrode of said device.

The examples and figures that follow are provided by way of non-limiting illustration of the field of the invention.

### Materials

Dextran T70 (Dex T70, MW=70,000 g/mol) and dextran T20 (Dex T20, MW=20,000 g/mol) from Pharmacosmos,
Methacrylic anhydride (94%),
Lithium phenyl-2.4,6-trimethylbenzoylphosphinate (LAP, >95%),
Phosphate buffer (PBS 10×): 1370 mm NaCl, 27 mm KCl, 100 mm Na2HPO4, 18 mm KH₂PO₄; pH=7.4),
NaH₂PO₄ (>99%),
D-glucose (>99%),
1,10-Phenantholine-5,6-dione (PLQ, 97%),
N,N-dimethylformamide (DMF, 99.9%), pig gelatin (gel strength 300, type A), agar, bovine serum albumin (BSA, >95%) from Sigma-Aldrich,
MWCNTs, (Ø=9.5 nm, length 1.5 μm, purity >95%) from Nanocyl,
PTFE filters from Millipore (JHWP, 0.45 μm pore size, Ø (filter)=46 mm), polydimethylsiloxane and its crosslinking agent (PDMS, Sylgard 184) from Dow,
Enzyme flavin adenine dinucleotide-dependent glucose dehydrogenase (FAD GDH) from Sekisui,
Phosphate buffers (PB 0.1M Na2HPO4; pH=7.4) and PBS (PBS1×: 137 mm NaCl, 2.7 mm KCl, 1 mm Na2HPO4, 1.8 mm KH₂PO₄; pH=7.4)

Artificial interstitial fluid (ISF): prepared by dissolving 22 g.1⁻¹ of BSA in PBS buffer and adjusting the pH to 7.4. The glucose solutions in PB, PBS and ISF were prepared at 1M by solubilizing the appropriate amount of glucose in PB, PBS or ISF and adjusting the pH to 7.4.

Artificial dermis (AD): Prepared as follows: Several solutions of gelatin (4% or 24% (w/w)) and of agar (1% (w/w)) were mixed in 40 ml of artificial ISF in the presence and absence of glucose (5 mmol.1⁻¹) in a 100 ml flask and heated to 80° C. for 30 minutes with stirring. Thereafter, the solution (AD4 or AD24, respectively comprising 4 or 24% gelatin) was poured into Petri dishes and allowed to cool overnight before use.

Skin phantom: It is composed of a fine Teflon membrane (Millipore PTFE filter, JHWP, pore size 0.45 μm, Ø (filter)=47 mm) to mimic the upper layer of the epidermis (mimics the dry horny layer), and the artificial dermis described above prepared in PBS with 22 g.1-1 of BSA in the presence or absence of glucose 5 mmol.1⁻¹ to mimic the lower layer of the dermis.

Electrochemical measurements were performed using a biological multipotentiostat VMP3 with EC-lab software or a Princeton Applied Research PARSTAT MC (PMC 1000/DC) running Versa Studio software with a three-electrode system consisting of
a pseudo-reference electrode made of Ag/AgCl wire (sat. KCl),
a platinum wire as counter-electrode and
a modified vitreous carbon electrode or a modified MWCNT-based electrode or the integrated MN-BS device described here as a working electrode.

The amperometric measurements were performed in a Faraday cage at room temperature using a Princeton Applied Research PARSTAT MC potentiostat (PMC 1000/DC) running Versa Studio software with a three-electrode system, consisting of an Ag/AgCl wire as pseudo-reference electrode, a platinum wire as counter-electrode, and the chosen working electrode.

### Example 1

### Preparation of Chemically Modified Dextran

Methacrylated dextrans were synthesized according to the method described in the application FR 2,114,492.

The detailed protocol is as follows: Dextran (5 g) was dissolved in 100 ml of distilled water (DW) in a beaker. After complete dissolution, various equivalents of methacrylic anhydride of 0.0625 to 0.5 equivalent relative to the hydroxyl functions of the dextran were added dropwise to the polymer solution in order to obtain the desired degree of substitution (DS) for the polymer, i.e. 9, 18, 37 or 62%. The pH was then adjusted with NaOH (3 mol.1⁻¹) in order to be around 9-11 throughout the reaction. The solution was stirred at ambient temperature for 1 hour. Finally, the modified dextran was dialyzed against distilled water for 1 week (12-14 kDa membrane) and lyophilized. The white solid was stored at −20° C. before use.

A PBS solution containing 20% (w/w) Dex-MA, with a DS of 9, 18, 37 or 62%, and 1% (w/w) LAP as photoinitiator was prepared in a 10 ml flask. 100 μl of solution were poured into a cylindrical Teflon mold measuring 6 mm in diameter (without microneedle cavity), and the solution was allowed to air dry for 24 hours. The polymer was crosslinked by a first irradiation of the back of the patch at 405 nm for 1 minute (P=75 mW/cm2), followed by demolding of the dry polymer cylinder. This yielded 4 dry photocrosslinked dextran methacylate materials (DS of 9, 18, 37 or 62%).

These 4 materials were characterized by a swelling test.

To do this, the 4 dried photocrosslinked Dex-MA materials were weighed (weight W0). The samples were placed in a flask containing PBS (1×) at ambient temperature (21-24° C.). They were withdrawn every hour, and their surface was quickly wiped off with a paper towel in order to remove excess water. Thereafter, the samples were immediately weighed (Wt) and replaced in PBS for the following measurement points. This operation was repeated until no further change in weight was detected. The swelling rate (SR) was calculated according to SR=(Wt−W0)/W0. Three experiments were carried out in parallel for each material.

Table 1 below shows the mean values of the swelling rates thus determined.

TABLE 1

| Sample Dex-MA | Swelling rate (%) |
|---|---|
| DS = 62% | 43.9 ± 2.2 |
| DS = 37% | 55.2 ± 2.0 |
| DS = 18% | 84.2 ± 1.5 |
| DS = 9% | 98.0 ± 2.5 |

The maximum swelling rate for each formulation was reached in about 1-2 hours. An increase in the SR is correlated with a decrease in the DS.

The hardness of each of these 4 materials was also characterized by a compression test. These compression tests were carried out using a TAXT.Plus texturometer (Stable Micro Systems, UK).

The compression rate was set at 0.5 mm·s−1 with a maximum compression force of up to 50 N. The compression modulus values, E, were calculated as the slope of the curve of applied force versus displacement. Table 2 shows these values.

TABLE 2

| Sample | Compression modulus (MPa) |
|---|---|
| Dex-MA DS = 62% | 159 ± 7 |
| Dex-MA DS = 37% | 167 ± 10 |
| Dex-MA DS = 18% | 161 ± 9 |
| Dex-MA DS = 9% | 155 ± 7 |

No alteration of the mechanical properties of the materials is observed. It is advantageous to note that the compression moduli of the photo-crosslinked dried DexMA materials are well above 10 kPa, the threshold value for piercing the skin.

### Example 2

### Preparation of a Microneedle (MN) Patch Coupled to a Working Bioelectrode According to the Invention

### A) Preparation of a Working Bioelectrode (WE) Composed of Carbon Nanotubes Coated with Electrochemical Mediator (PLQ) and Enzyme (FADGDH)

66 mg of MWCNT were dispersed in 66 ml of DMF in a 100 ml flask and placed in a water bath under sonication for 1 h 30 min. The suspension was filtered through a PTFE filter using a vacuum pump, washed with distilled water and left for 2 hours under a hood. After filtration, the resulting carbon nanotube “paper” was allowed to dry at ambient temperature against another PTFE filter for 24 hours. The carbon nanotube “paper” was delicately detached from the filter paper and cut into individual electrodes of Ø=4 or 6 mm. The MWCNTs were then modified by a first addition of 9 and 20 μl, respectively, of a PLQ solution (5 mm) in an acetone/H₂O mixture (volume ratio 1:1) and allowed to dry for 10 minutes. 13.5 and 30 μl, respectively, of a solution of FADGDH in phosphate buffer PB (10 mg·ml-1) were deposited on the surface of the modified electrode and allowed to dry for a few hours. Finally, the electrical contact was made using a metal wire fixed to the rear of the bioelectrode with carbon paste and allowed to dry for 2 hours. The back of the electrode was insulated with silicone paste and allowed to dry for a few hours before use.

### B) Preparation of the Polydimethylsiloxane (PDMS) Mold for the MN Patch

A master mold was produced from aluminum by micro-milling. The aluminum mold was composed of a cylindrical network of 7 mm with 5×5 (25) microneedles in the shape of pencils with heights of 800, 1000 and 1200 μm respectively, a width at the base of 400 μm, and an edge-to-edge distance between the needles of 400 μm. A reverse mold was prepared by casting a mixture of PDMS and its curing agent (ratio of 10:1) on the main mold, under vacuum to remove air bubbles, and then hardened at 100° C. for 2 hours. Finally, the master and reverse molds were cooled under air for a few hours, and the PDMS mold was gently detached from the aluminum mold.

### c) Preparation of the MN Patch with Integration of the Working Electrode

A PBS solution containing 20% (w/w) Dex-MA, with a DS of 9, 18, 37 or 62%, and 1% (w/w) LAP as photoinitiator was prepared in a 10 ml flask. 100 μl of solution were poured into a PDMS mold. The mold was placed in an aluminum support connected to a vacuum pump, and a reduced pressure (1-10 mbar) was applied for 2 hours to fill the mold tips. The working electrode (WE) prepared at a) was then integrated into the MN patch by inserting the electrode inside the polymer solution so that the surface of the electrode was close to the rear of the tips, and the rear of the electrode was exposed to allow the electrical connection 9. The MN patch with the integrated electrode WE was then allowed to dry for 24 hours. The polymer was crosslinked by a first irradiation of the back of the patch at 405 nm for 1 minute (P=75 mW/cm2), followed by demolding of the device and a last irradiation at 405 nm for 1 minute on the needle face of the patch. Finally, the electrical connection was made by bonding an Ag/Cu wire with carbon paste to the back of the electrode.

### Example 3

### Test of the Efficacy of the MN Device with Integrated MWCNT-Based Working Electrode for Electro-Oxidation and Glucose Detection

The MN device considered is a crosslinked Dex-MA microneedle patch (DS=9%) incorporating a working electrode based on MWCNT with immobilized PLQ and FADGDH and prepared according to example 2c).

Cyclic voltammograms, shown in FIG. 2, were recorded in the absence and presence of glucose in a phosphate buffer solution prepared at 0.1 M, pH=7.4 (PB) in air, in a 3-electrode cell using the MN device as a working electrode, a platinum wire as counter-electrode, and a chlorinated silver wire (Ag/AgCl) as reference electrode. In the absence of glucose (dotted line), the redox signal of the PLQ is observed with a redox activity typical of quinone (dominant low potential redox couple around E1/2=−0.24 V versus pseudo-reference Ag/AgCl). In the presence of 0.1 M glucose (solid line), a strong increase in the current is observed with a low attractive trigger potential of about-0.3 V compared with the Ag/AgCl pseudo-reference.

It appears that the process of integration of the bioelectrode into the hydrogel matrix did not significantly disrupt the bioelectrocatalytic oxidation of glucose. This same efficiency of bioelectrocatalytic oxidation was verified with microneedle Dex-MA patches of DS of 18, 37 and 62%.

### Example 4

### Use of a Device According to the Invention for Continuous Measurement of Blood Glucose in a Two-Layer Artificial Skin Phantom

Continuous glucose monitoring was performed with the integrated crosslinked Dex-MA MN electrode (DS=37%) prepared according to example 2 in a two-layer artificial skin phantom as detailed above in the section Materials and methods.

The amperometric measurements were carried out as described also in the section Materials and methods. The counter-electrodes and the reference electrodes were here inserted directly into the skin phantom (no integration with the MN patch). The needles of the crosslinked Dex-MA (DS=37%) MN device integrating a bioelectrode of the working electrode type based on MWCNTs coated with PLQ and FADGDH, as prepared according to example 2c), pierced the two-layer artificial skin phantom (24%) using a penetration force of 10 N, and a constant force of about 1 to 2 N to hold the needles inside the artificial dermis.

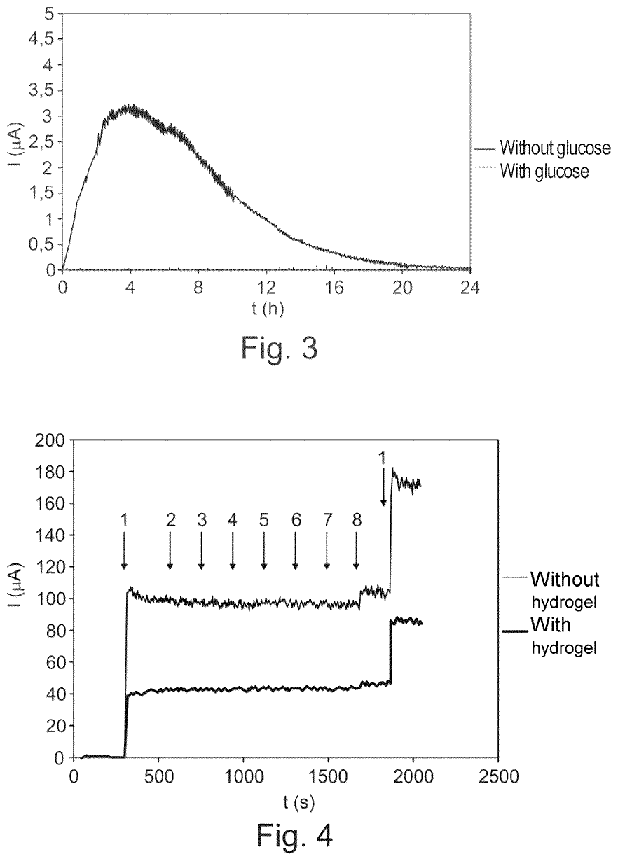

Immediately after insertion and concomitant application of −0.1 V, the recorded current increased to approximately 3 μA (FIG. 3). In the absence of glucose, the current does not exceed 0.1 μA. Maximum catalytic current was reached at about 4 hours. A reduction in current, typical of these 2nd generation glucose enzyme electrodes, was then obtained until about 22 hours, when electrode performance decreased to less than 2% of maximum catalytic current.

### Example 5

### Device for Glucose Detection with an MWCNT-Based Bioelectrode without Hydrogel Versus a Device According to the Invention with Crosslinked Hydrogel (DexMA 37%): Characterization in the Presence of Different Interfering Species in the Artificial ISF

The amperometric measurement with the glucose electrode considered in the previous example can be performed at different potentials, the applied potential having an impact on factors such as the contribution of the electrochemical interference and also the bioelectrocatalysis which affects the performance of the sensor.

FIG. 4 shows a chronoamperogram recorded at −0.1 V which shows, firstly, a glucose sensor signal (oxidation current) after addition of 5 mmol 1⁻¹ of physiologically relevant glucose. Successive additions of potential interferents were then made. The current remained virtually unchanged with the addition of acetaminophen, cholesterol, urea, lactate, galactose and uric acid (UA) (2-7). Uric acid and acetaminophen are known to be oxidized at potentials that interfere with glucose sensors and are therefore known as common electrochemical interferents. On the other hand, the glucose electrode signal (oxidation current) changed significantly with the addition of ascorbic acid (AA) (8), corresponding to the electrochemical oxidation of the compound at the working potential of −0.1 V compared to the pseudo-reference Ag/AgCl. Nevertheless, this current only implies an interference rate of 6.8%, which is very acceptable. Finally, a second addition of 5 mmol of glucose L−1 was carried out (1), which again led to an increase in the current, showing the robustness of the MWCNT-based biosensor device with respect to the presence of interfering species. However, this current is less than the current resulting from the first addition of glucose. The same experiment was carried out on devices having a coating based on crosslinked Dex-MA DS=37% in accordance with the invention. Similarly to the devices without hydrogel, an increase in current is obtained with the first addition of glucose, followed by a virtually unchanged current with the addition of acetaminophen, cholesterol, urea, lactate, galactose and uric acid (UA) (2-7); an oxidation of ascorbic acid is observed upon its addition, due to its oxidation at this working potential, implying an interference rate of only 4.9%, which shows the beneficial effect of the hydrogel with respect to interfering species. Finally, a last increase in the current is observed following the last addition of glucose, the value of which addition is very close to that obtained during the first addition, showing the protective and stabilizing effect of the hydrogel to interfering species with respect to the device.

### Example 6

### Characterization of the Stability of an MN Patch with an Integrated Bioelectrode According to the Invention Versus an Electrode Immobilized on the Back of the Patch

A PBS solution containing 20% (w/w) Dex-MA with a DS of 37% and 1% (w/w) LAP as photoinitiator was prepared in a 10 ml flask. The solution was cast into the PDMS mold prepared in example 1. The mold was placed in an aluminum support connected to a vacuum pump, and a reduced pressure (1-10 mbar) was applied for 24 hours to fill the mold tips and dry the polymer. The dry polymer was then crosslinked under UV-vis at 405 nm for 1 minute (P=75 mW/cm2). The MWCNT-based electrode (prepared in example 2) was then integrated by depositing a polymer layer containing 20% (w/w) Dex-MA, with a DS of 37%, and 1% (w/w) LAP on the electrode and gluing the electrode to the back of the patch. The MN patch thus obtained was then exposed again to UV-vis at 405 nm for 1 minute in order to crosslink the adhesive layer.

This device not in accordance with the invention and the device according to the invention, and prepared using a Dex-MA DS of 37% in example 2, were then analyzed by chronoamperometry at −0.1V by immersing the hydrogel part of the device in a PB solution with stirring for 1 hour min, followed by an addition of 5 mm glucose. However, after 24 hours, no catalytic current was observed with the MN patch not in accordance with the invention. Partial detachment of the electrode was also observed.

1. A device for transdermal electrochemical measurements, the device comprising:
at least one polymeric support having a surface configured for contact with skin;
at least one array of polymeric microneedles integral with the support and projecting outward from the surface of the support configured for contact with the skin; and
at least one porous bioelectrode comprising at least one biologically active species, immobilized on the surface of a conductive material,
wherein the polymeric microneedles and at least the surface of contact of the support with the skin are formed of a biocompatible crosslinked hydrogel, non-electron-conductive in the dry state and electrolyte-conductive upon contact with an aqueous fluid, and
wherein the at least one porous bioelectrode is arranged in contact with the hydrogel configured to swell upon contact with the aqueous fluid, is free of direct contact with the skin, and is at least partially interpenetrated by crosslinked hydrogel.

2. The device of claim 1, wherein the at least one porous bioelectrode is integrated in the crosslinked hydrogel constituting the support which is in direct contact with the rear of the polymeric microneedles.

3. The device of claim 1, wherein the hydrogel is free of any metallic constituent and electron-conductive polymer.

4. The device of claim 1, wherein the hydrogel is free of any biologically active species other than that immobilized on the at least one porous bioelectrode.

5. The device of claim 1, wherein the polymeric microneedles are not hollow.

6. The device of claim 1, wherein the biologically active species is an enzyme, optionally combined with a molecule that facilitates electron transfer between the enzyme and the at least one porous bioelectrode.

7. The device of claim 1, wherein the at least one porous bioelectrode is formed of a nano-structured or micro-structured conductive material.

8. The device of claim 1, characterized in that it comprises wherein the at least one porous bioelectrode is an enzymatic bioelectrode.

9. The device of claim 1, wherein the hydrogel is obtained by crosslinking one or more biopolymers, one or more synthetic polymers, or mixtures thereof, wherein the one or more biopolymers and the one or more synthetic polymers are optionally chemically modified to be crosslinkable.

10. The device claim 9, wherein the one or more biopolymers is at least one selected from the group consisting of a polyhydroxy acid, a polysaccharide, a protein, and a peptide.

11. The device claim 9, wherein the one or more biopolymers is at least one selected from the group consisting of an alginate, hyaluronic acid, carboxymethylcellulose, chitosan, dextran, and a derivative thereof.

12. The device of claim 1, wherein the hydrogel is derived from the crosslinking of at least one dextran polymer modified by units chosen from acrylate, methacrylate, alkenyl, and alkynyl.

13. The device of claim 1, of which is a transdermal microneedle patch.

14. The device of claim 1, comprising at least one auxiliary electrode, distinct or not from the at least one porous bioelectrode.

15. The device of claim 1, further comprising an electrochemical detection device attached to the at least one porous bioelectrode via electrical connections.

16. A method for preparing a device as claimed in any one of the preceding claims the device of claim 1 by micro-molding, the method comprising:
crosslinking at least one biopolymer to form the crosslinked hydrogel,
wherein the at least one porous bioelectrode is integrated by being placed in contact with the biopolymer prior to or simultaneously with its crosslinking.

17. A method for analyzing an analyte in an interstitial fluid, the method comprising:
contacting the interstitial fluid with the polymeric microneedles of the device of claim 1.

18. A method for detecting and/or assaying at least one analyte in an interstitial fluid, the method comprising:
contacting the polymeric microneedles of the device in claim 1 with the interstitial fluid under conditions that are conducive to the swelling of the constituent hydrogel of the polymeric microneedles by this fluid and to the diffusion of this fluid, by a difference in osmotic pressure and/or capillarity, as far as the at least one porous bioelectrode.
